(19) United States
(12) Patent Application Publication
(10) Pub. No.: US 2025/0224369 A1
(43) Pub. Date: Jul. 10, 2025

(54) CAPILLARY ELECTROPHORESIS METHODS FOR THE SIMULTANEOUS SEPARATION OF NUCLEIC ACIDS OF VARYING LENGTHS

(71) Applicant: DH TECHNOLOGIES DEVELOPMENT PTE. LTD., Singapore (SG)

(72) Inventors: Mervin GUTIERREZ, West Covina, CA (US); Fang WANG, Chino Hills, CA (US); Mario PULIDO, La Habra, CA (US)

(21) Appl. No.: 18/850,799

(22) PCT Filed: Mar. 24, 2023
(86) PCT No.: PCT/IB2023/052950

§ 371 Date: Sep. 25, 2024

Related U.S. Application Data
(60) Provisional application No. 63/324,859, filed on Mar. 29, 2022.

(51) Int. Cl. G01N 27/447; C12Q 1/6876

(52) U.S. Cl. CPC G01N 27/44791; C12Q 1/6876; G01N 27/44726; G01N 27/44743; C12Q 2600/166

(57) ABSTRACT

The disclosed technology provides methods for analyzing biomolecules over a wide range of molecular weights.

### RELATED APPLICATIONS

The present patent application claims the priority benefit of U.S. Provisional Patent Application Ser. No. 63/324,859, filed Mar. 29, 2022, the content of which is hereby incorporated by reference in its entirety into this disclosure.

### BACKGROUND

CRISPR (clustered regularly interspaced short palindromic repeats) is an immunological defense mechanism employed by bacteria against invading pathogens. Two RNA molecules are essential for CRISPR: Cas9 (CRISPR associated protein 9), an RNA-guided DNA endonuclease, and single guide RNA (sgRNA). Due to its ability to perform site directed DNA cleavage and to trigger homologous recombination, Cas9 has been utilized as a gene editing tool to introduce gene inactivation and genome modification. CRISPR can improve existing therapies in the pharmaceutical industry, help find drug targets, and test drug candidates.

RNA-vaccines and CRISPR-based gene editing, for example, are two recent breakthroughs for combating SARS CoV-2 and with great potential for treating many genetic diseases, respectively. However, RNA vaccine and CRISPR reagent manufacturers often face challenges with the accurate characterization and quantification of differently sized RNA molecules, impurities, and/or degraded RNA species found as part of vaccines or personalized medicinal products. During the development of RNA therapeutics, it is often advantageous to co-deliver the sgRNA and the Cas9 mRNA. Purity analysis of these two molecules currently requires two methods: denaturing agarose gel electrophoresis for Cas9 mRNA and denaturing polyacrylamide gel electrophoresis for sgRNA. Therefore, methods allowing for the simultaneous analysis of a broad range of nucleic acid lengths would be advantageous.

### SUMMARY

The inventors have recognized the need to co-analyze nucleic acids, such as sgRNA and Cas9 mRNA, using the same polymer matrix. The claimed and described capillary electrophoresis methods offer high resolution and provide accurate characterization and quantification of differently sized nucleic acids using capillary electrophoresis run without diluting the polymer matrix. In an aspect, the disclosure provides a method for analyzing nucleic acids of differing sizes, the method comprising loading at least two nucleic acids of differing sizes on a capillary electrophoresis (CE) capillary wherein the CE capillary is filled with a buffer comprising a polymer matrix; applying a voltage to the CE capillary to simultaneously separate the nucleic acids; and detecting the separated nucleic acids with a detector, wherein at least one nucleic acid is shorter than about 200 nucleotides and at least one nucleic acid is longer than about 4000 nucleotides.

In another aspect, the at least two nucleic acids are loaded onto the CE capillary using hydrodynamic injection or electrokinetic injection. In a further aspect, the at least two nucleic acids are loaded onto the CE capillary using hydrodynamic injection

In another aspect, at least one nucleic acid is shorter than about 150 nucleotides, alternatively shorter than about 100 nucleotides, alternatively shorter than about 50 nucleotides, alternatively shorter than about 25 nucleotides.

In another aspect, at least one nucleic acid is longer than about 4200 nucleotides, alternatively longer than about 4500 nucleotides, alternatively longer than about 4800 nucleotides, alternatively longer than about 5000 nucleotides, alternatively longer than about 5500 nucleotides, alternatively longer than about 6000 nucleotides, alternatively longer than about 6500 nucleotides, alternatively longer than about 7000 nucleotides, alternatively longer than about 7500 nucleotides, alternatively longer than about 8000 nucleotides, alternatively longer than about 8500 nucleotides, alternatively longer than about 9000 nucleotides, alternatively longer than about 9500 nucleotides, alternatively longer than about 10000 nucleotides.

In another aspect, the at least two nucleic acids are selected from the group consisting of deoxyribonucleic acid (DNA), ribonucleic acid (RNA), single-stranded DNA (ssDNA), double-stranded DNA (dsDNA), microRNA (miRNA), messenger RNA (mRNA), and RNA fragments. In a further aspect, the at least two nucleic acids are single stranded RNA fragments. In yet a further aspect, the single stranded RNA fragments are sgRNA and Cas9mRNA.

In another aspect, the at least two nucleic acids are loaded on the CE capillary as a mixture or are loaded sequentially.

In another aspect, the polymer matrix is selected from the group consisting of crosslinked polymer, linear polymers, slightly branched polymers, linear polyacrylamide, polyethylene oxide, polyethylene glycol and dextran.

In another aspect, the method further comprises adding a fluorescent dye to the at least two nucleic acids, the polymer matrix and/or to a buffer disposed within the CE capillary, wherein the fluorescent dye binds the nucleic acids resulting in fluorescently labeled nucleic acids. In a further aspect, the fluorescent dye is a cyanine-based dye. In yet a further aspect, the cyanide-based dye is selected from the group consisting of Cy2, Cy3, Cy3B, Cy3.5, Cy5, Cy5.5, Cy7, SYBR Green I, SYBR Green II, PicoGreen, Thiazole orange, and Oxazole yellow.

In another aspect, the method further comprises heating at least one of the at least two nucleic acids prior to loading the nucleic acids on the CE capillary. In a further aspect, the nucleic acid is heated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C. In yet a further aspect, the nucleic acid is heated for at least 2 minutes, alternatively at least 3 minutes, alternatively at least 4 minutes, alternatively at least 5 minutes.

In another aspect, the method further comprises cooling the nucleic acid after heating. In a further aspect, the nucleic acid is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

In another aspect, at least one of the at least two nucleic acids is diluted with a sample solution, water, or combinations thereof prior to loading on the CE capillary.

In another aspect, the at least two nucleic acids are separated using capillary gel electrophoresis or capillary electrochromatography.

In another aspect, the detector is a UV detector or fluorescence detector. In a further aspect, the detector is a laser-induced fluorescence (LIF) detector, a lamp-based fluorescence detector, or a native fluorescence detector. In another aspect, detecting the nucleic acid utilizes a fluorescence detector.

In another aspect, the method results in increased peak efficiency and/or high-resolution.

One aspect of the disclosure is a kit for analyzing at least two nucleic acids, wherein at least one nucleic acid is shorter than about 200 nucleotides and at least one nucleic acid is longer than about 4000 nucleotides, the kit comprising a CE capillary, a cartridge comprising at least one capillary, or a capillary electrophoresis chip, a buffer comprising a polymer matrix, and instructions for use.

In another aspect, the kit further comprises a fluorescent dye, a regenerating solution, a diluent, and/or an ssRNA ladder.

One aspect of the disclosure is a kit for analyzing at least two nucleic acids, wherein at least one nucleic acid is shorter than about 200 nucleotides and at least one nucleic acid is longer than about 4000 nucleotides, the kit including a buffer comprising a polymer matrix and instructions for use. In another aspect, the kit further includes a fluorescent dye, a regenerating solution, a diluent, and/or an ssRNA ladder.

Other aspects and features of the present disclosure will become apparent to those ordinarily skilled in the art upon review of the following description of specific embodiments of the disclosure in conjunction with the accompanying figures.

### BRIEF DESCRIPTION OF THE FIGURES

Embodiments of the present disclosure will now be described, by way of example only, with reference to the attached Figures.

FIG. 1 illustrates a workflow diagram for co-analysis of small and large RNA fragments according to one aspect of the disclosure.

FIG. 2 shows an overlay electropherograms (or “e-grams”) sgRNA and ssRNA ladder analyzed according to one aspect of the disclosure.

FIG. 3 shows the overlay of three injections of Cas9 mRNA sample analyzed for purity according to one aspect of the disclosure.

FIG. 4 shows the overlay of three injections of a sgRNA analyzed according to one aspect of the disclosure.

FIG. 5 shows the overlay of three injections of Cas9 mRNA analyzed according to one aspect of the disclosure.

FIGS. 6A and 6B illustrate an intra-capillary analysis (n=3 injections) from a mixed sample containing Cas9 mRNA and sgRNA (red, green, and blue traces) analyzed using a BioPhase 8800 System (FIG. 6A) and a PA 800 Plus Pharmaceutical Analysis System (FIG. 6B).

FIGS. 7A-7C illustrates the BioPhase 8800 system operation sequence for the conditioning (FIG. 7A), optimized pressure-based separation method with a water-plug step prior to sample injection (FIG. 7B), and shut down methods used (FIG. 7C) according to one aspect of the disclosure.

FIGS. 8A-8C illustrates PA 800 Plus Pharmaceutical Analysis separation method for Cas9 mRNA and HPRT1 sgRNA analysis. FIG. 8A shows the current, cartridge temperature, and sample storage temperature settings. FIG. 8B illustrates the LIF detector settings highlighted by an excitation wavelength of 488 nm and an emission wavelength of 520 nm. FIG. 8C shows the pressure-based separation conditions as used for the BioPhase 8800 system.

FIG. 9 shows a comparison overlay e-grams for the ssRNA ladders separated at different temperature conditions. As the temperature decreases the migration time, peak resolution for all marker are affected. Details and quantitative comparison are listed in the following FIG. 10. From bottom to the top in the e-grams, the corresponding capillary cartridge temperatures are 25, 30, 35, 40° C.

FIGS. 10A-10D shows a quantitative analysis of ssRNA marker separation performance. Scatter plot of theoretical plates, peak resolution, peak width and migration time for all the RNA markers at seven different temperatures ((25: circle, 30: squares, 35: diamond, 40: upward-triangle, 45: right-pointing triangle, 50: left-pointing triangle, 55: downward-triangle)). FIG. 10A shows a scatterplot of migration time vs peak. FIG. 10B shows a scatterplot of theoretical plates vs peak. FIG. 10C shows a scatterplot of resolution vs peak. FIG. 10D shows a scatterplot of width vs peak.

FIG. 11 shows separation results with hydrodynamic injection method. Electropherogram of ssRNA ladder analyzed with hydrodynamic injection (1 psi for 5 seconds). The inset shows the y-axis zoom in for markers below 300 bases. The other condition used include 30° C. capillary temperature 200 V/cm field strength and with 20/30 cm 50 m ID BFS capillary.

FIG. 12 shows separation results with electrokinetic injection method. Electropherogram of ssRNA ladder analyzed with electrokinetic injection (5 kv for 3 seconds). The inset shows the y-axis zoom in for markers below 300 bases. The other condition used include 30° C. capillary temperature, 200 V/cm field strength and with 20/30 cm 50 m ID BFS capillary.

FIG. 13 shows a comparison of theoretical plates for each ssRNA markers using EKI and HDI. The other condition used include EKI (5 kv for 3 seconds) and HDI (1 psi for 5 seconds). 30° C. capillary temperature 200 V/cm field strength and with 20/30 cm 50 m ID BFS capillary.

FIG. 14 shows assay repeatability using EKI injection method. Electropherograms overlay of 16 ssRNA ladder analyzed with EKI injection (5 kv for 3 seconds). The other condition used include 30° C. capillary temperature 200 V/cm field strength and with 20/30 cm 50 m ID BFS capillary.

FIG. 15 shows assay repeatability using hydrodynamic injection method. Electropherograms overlay of 16 ssRNA ladder analyzed with hydrodynamic injection (1 psi for 5 seconds). The other condition used include 30° C. capillary temperature 200 V/cm field strength and with 20/30 cm 50 m ID BFS capillary.

### DETAILED DESCRIPTION

It is to be understood that this disclosure is not limited to the particular methodology, protocols, and reagents described herein and as such may vary. It is also to be understood that the terminology used herein is for the purpose of describing particular embodiments only and is not intended to limit the scope of the present disclosure or the appended claims.

Unless otherwise defined, all technical and scientific terms used herein have the same meaning as commonly understood by one of ordinary skill in the art to which the methods described herein belong.

The singular form “a”, “an” and “the” include plural referents unless the context clearly dictates otherwise. These articles refer to one or to more than one (i.e., to at least one). The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

The term “about” as used in connection with a numerical value throughout the specification, and the claims denotes an interval of accuracy, familiar and acceptable to a person skilled in the art. In general, such interval of accuracy is +/−10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “exemplary” means serving as a non-limiting example, instance, or illustration. As utilized herein, the terms “e.g.,” and “for example” set off lists of one or more non-limiting aspects, examples, instances, or illustrations.

The disclosure generally relates to capillary electrophoresis methods and kits for analyzing nucleic acids of different sizes. In some aspects, this method comprises loading at least two nucleic acids of differing sizes on a capillary electrophoresis (CE) capillary using hydrodynamic injection, wherein the CE capillary is filled with a buffer comprising a polymer matrix; applying a voltage to the CE capillary to simultaneously separate the nucleic acids; and detecting the separated nucleic acids with a detector, wherein at least one nucleic acid is shorter than about 200 nucleotides and at least one nucleic acid is longer than about 4000 nucleotides.

“Polynucleotide(s)”, “nucleic acid sequence”, “nucleotide sequence”, and “nucleotide(s)” can be used interchangeably and refer to a continuous sequence of any type of nucleic acid molecule(s). Non-limiting examples of nucleic acids include deoxyribonucleic acid (DNA), ribonucleic acid (RNA), mixed RNA/DNAs, single-stranded DNA (ssDNA), double-stranded DNA (dsDNA), microRNA (miRNA), messenger RNA (mRNA), and RNA and/or DNA fragments, impurities, or degraded molecules.

In some aspects, the nucleic acids are Cas9 mRNA and sgRNA.

In some aspects, at least one of the nucleic acids analyzed has a length of about 200 nucleotides or less. In some non-limiting examples, at least one nucleic acid is shorter than about 150 nucleotides, alternatively shorter than about 100 nucleotides, alternatively shorter than about 50 nucleotides, or alternatively shorter than about 25 nucleotides.

In some aspects, at least one of the nucleic acids analyzed has a length of about 4000 nucleotides or greater. In some non-limiting examples, at least one nucleic acid is longer than about 4200 nucleotides, alternatively longer than about 4500 nucleotides, alternatively longer than about 4800 nucleotides, alternatively longer than about 5000 nucleotides, alternatively longer than about 5500 nucleotides, alternatively longer than about 6000 nucleotides, alternatively longer than about 6500 nucleotides, alternatively longer than about 7000 nucleotides, alternatively longer than about 7500 nucleotides, alternatively longer than about 8000 nucleotides, alternatively longer than about 8500 nucleotides, alternatively longer than about 9000 nucleotides, alternatively longer than about 9500 nucleotides, or alternatively longer than about 10000 nucleotides.

Nucleic acids of the present disclosure may be extracted from organic, biochemical, biological material or biomolecules, for example, micelles, microparticles, nanoparticles (e.g., lipid nanoparticles LNPs), tissue, cells, blood, microbes, bacterial or viral vectors, proteins, peptides, or polypeptides. “Polypeptide”, “protein”, and “peptide” can be used interchangeably, and refer to polymers of amino acids of any length.

As used herein, “capillary” refers to a channel, tube, or other structure capable of supporting a volume of separation medium for performing electrophoresis. Capillary geometry can vary and includes structures having circular, rectangular, or square cross-sections, channels, grooves, plates, and the like that can be fabricated by technologies known in the art. Capillaries of the present disclosure can be made of materials such as, but not limited to, silica, fused silica, quartz, silicate-based glass such as borosilicate glass, phosphate glass, alumina-containing glass, and other silica-like materials. In some aspects, the methods can be adapted and used in any generally known electrophoresis platform, such as, for example, electrophoresis devices comprising single or multiple microfluidic channels, etched microfluidic capillaries, as well as slab gel and thin-plate gel electrophoresis.

In some aspects, the capillary is an uncoated capillary.

In some aspects, the capillary is a coated capillary. For example, a capillary can be coated to shield or minimize electrostatic interactions. Shielding can comprise non-permanent, replaceable polymeric hydrophilic coatings that adsorb to the capillary surface or permanent hydrophilic coatings comprising for example linear polyacrylamide or polyvinylalcohol that covalently bind the capillary surface.

In some aspects, the nucleic acids are loaded using hydrodynamic injection (HDI). HDI typically comprises the use of pressure to drive a small sample volume into the capillary. Alternatively, electrokinetic injection (EKI) is where an electric field is used to drive only the charged species of the sample into the capillary. When analyzing at least two nucleic acids, wherein one of the nucleic acids is smaller than 300 nucleotides, nucleic acids loaded using HDI provide several benefits, including improved resolution and/or peak efficiency compared to EKI. The difference between these two injections is that the HDI is representative of all sample components.

In contrast, the EKI injection introduces a bias because it preferentially injects species in the sample with higher mobility. Increased peak widths of nucleic acids between 50 nucleotides and 300 nucleotides are observed when using EKI. As such, EKI could negatively impact the purity assessment of fragments size between 50 nucleotides and 300 nucleotides. Therefore, a pressure injection should be considered if the analyte of interest is below 300 nucleotides in length. Conversely, EKI is inherently a stacking technique because it induces a pre-concentration of the sample band during the injection, resulting in a five-fold increase in signal response. However, it is possible to impart stacking capability to the HDI by simply injecting a small water plug before the injection of the sample. This technique significantly improves peak shape and resolution for the ssRNA fragments smaller than 300 nucleotides.

In some aspects, the at least two nucleic acids are loaded on the CE capillary as a mixture. In a non-limiting example, a solution of nucleic acids to be analyzed can be prepared by dissolving a nucleic acid of interest into a solvent. Multiple nucleic acid solutions can be prepared based on the number of nucleic acids to be analyzed. When two nucleic acids are analyzed, an aliquot or portion of a first nucleic acid solution can be mixed with an aliquot or portion of a second nucleic acid solution, and the resulting mixture can be loaded onto the CE capillary. In another aspect, a first nucleic acid solution and a second nucleic acid solution are loaded sequentially on the CE capillary.

The CE capillary may be filled with a buffer comprising a polymer matrix or gel buffer prior to applying a separation voltage and/or loading the nucleic acids. In some aspects, the buffer comprising a polymer matrix or gel buffer is placed into a buffer vial(s). These buffer vials may be placed into buffer trays. In some aspects, the buffer comprising a polymer matrix or gel buffer may comprise additional components to facilitate the separation of the nucleic acids. Non-limiting examples of a suitable polymer matrix include crosslinked polymer, linear polymers, slightly branched polymers, linear polyacrylamide, polyethylene oxide, polyethylene glycol, and dextran.

In some aspects, the disclosed methods allow for the simultaneous analysis of at least two nucleic acids of differing sizes. In some aspects, nucleic acids comprising a relatively large size difference, such as sgRNA and Cas9 mRNA, are separated and analyzed using one CE run and without diluting the polymer matrix. This polymer matrix combined with hydrodynamic injection unexpectedly results in ultra-high resolution separation technology allowing for simultaneous analysis of small and large nucleic acids with excellent repeatability.

Because nucleic acid molecules, such as RNAs, are highly unstable and fragile, their stability and handling in vitro are always a concern. As further described and illustrated below, the nucleic acid profiles are consistent between different runs and back-to-back injections, indicating that the disclosed methods do not appear to damage, change, or alter the nucleic acids in the samples.

As noted above, nucleic acids are susceptible to fluctuations, and RNA, in particular, is chemically more transient than other nucleic acids. However, as further described and illustrated below, there were no high molecular weight complexes that were significantly larger than the nucleic acids being analyzed, indicating that the disclosed methods did not induce the formation of any new nucleic acid molecules.

In some aspects, a fluorescent dye is added to the nucleic acids, a polymer matrix, a buffer, or both the polymer matrix and the buffer, which results in a fluorescently labeled nucleic acid. In these aspects, the fluorescent dye is a cyanine-based dye. Cyanine-based dyes of the disclosure include, not are not limited to, Cy2, Cy3, Cy3B, Cy3.5, Cy5, Cy5.5, Cy7, SYBR Green I, SYBR Green II, PicoGreen, Thiazole orange, and Oxazole yellow. In exemplary aspects, the fluorescently labeled dye is SYBR Green I or SYBR Green II.

In an aspect, the nucleic acids are diluted prior to being loaded onto the CE capillary. The nucleic acids may be diluted with, for example, a sample solution, water, or a combination thereof. The sample solution may be a sample loading solution, and in some aspects, the sample solution is formamide. The water may be deionized water, CE-grade water, or nuclease-free water.

In some aspects, the methods optionally include heating at least one nucleic acid and/or optionally cooling the heated nucleic acid prior to loading on the CE capillary.

When the method includes heating the nucleic acid, the nucleic acid may be heated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., or alternatively at a temperature of about 70° C. In example embodiments, the heating comprises denaturing the nucleic acid(s).

The nucleic acid may be heated for at least 2 minutes, alternatively at least 3 minutes, alternatively at least 4 minutes, or alternatively at least 5 minutes.

After heating, in some aspects, the nucleic acid is cooled. The nucleic acid may be cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, or alternatively at least about 60 minutes.

In an aspect, a voltage is applied to the CE capillary to separate the nucleic acids. Different voltages may be applied to optimize the resolution of the separation. The voltage may be optimized depending on the size and the required resolution of the analytes.

In the methods described herein, when a voltage is applied to the CE capillary, the nucleic acids are mobilized based on overall charge and migrate towards a detector. During migration, nucleic acids are differentiated by size, with the larger nucleic acid(s) having a longer migration time.

In some aspects, the field strength during mobilization of the nucleic acids is about 200 to about 1000 V/cm, alternatively about 200 to about 750 V/cm, alternatively about 200 to about 500 V/cm, alternatively about 200 to about 250 V/cm, alternatively about 200 V/cm.

As described herein, the nucleic acids may be separated using capillary gel electrophoresis or capillary electrochromatography. In an aspect, the separation is done using capillary gel electrophoresis (CGE), which separates samples by size and detects the nucleic acids using a fluorescent dye that binds to the nucleic acids.

The separated nucleic acids may be detected using a detector. The detector can be a UV detector or a fluorescence detector, such as a laser-induced fluorescence (LIF) detector, a lamp-based fluorescence detector, or a native fluorescence detector. The desired quantitation sensitivity will determine the type of detector used. LIF detection offers the benefit of about an increase in sensitivity, yet it also requires additional sample manipulation.

In some aspects, detecting the nucleic acids produces a set of corresponding values that can be used to quantify or otherwise analyze the nucleic acids. In some aspects, these corresponding values can be plotted on an electropherogram.

An “electropherogram” (or “e-gram”) refers to a series of peaks that can be converted to determine the size and/or quantity of a sample. Peaks are integrated for area as a measure of quantity, and can be corrected for mobility differences between different sized peaks. In some aspects, a nucleic acid ladder comprising nucleic acid fragments of known size can be run before, during, or after sample(s) of interest.

In some aspects, the disclosure relates to methods and kits for differentiating single stranded RNA fragments of various sizes in the range of about 50 nucleotides to about 9,000 nucleotides with high-resolution data.

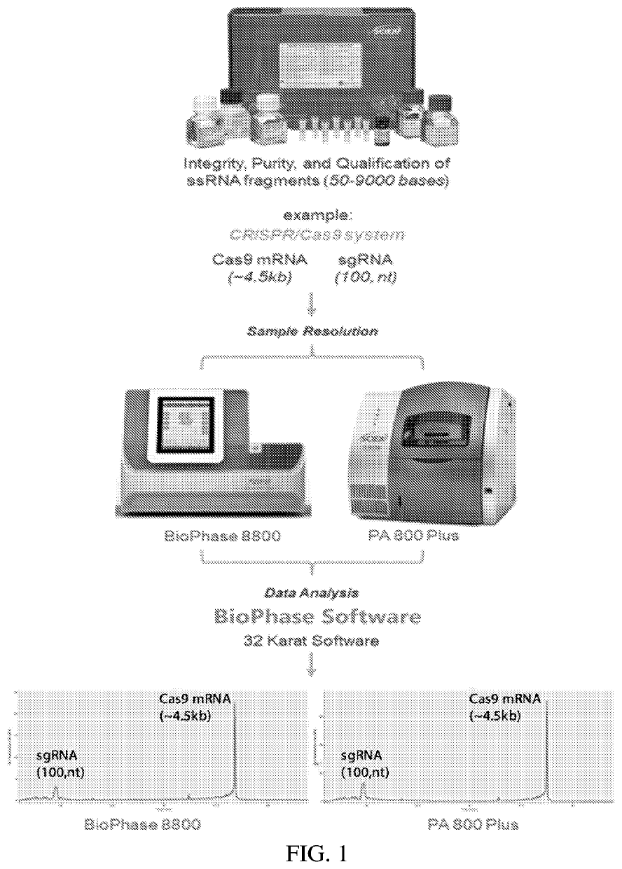

FIG. 1 illustrates a CRISPR/Cas9 system workflow using two different instruments. As shown in FIG. 1, the workflow enabled high resolution of relatively small (100 nts) (e.g., sgRNA) and relatively large (˜4.5 kb) (e.g., Cas9mRNA) RNA fragments. In a CRISPR/Cas9 workflow, the guide RNA provides sequence-specificity and targets the Cas9 nuclease to the site of gene editing, where the Cas9 nuclease performs double-stranded DNA cleavage. The guide RNA occurs naturally as a two-molecule complex consisting of a target-specific crRNA (CRISPR RNA) bound to a tracrRNA (trans-activating crRNA) that directs the binding of RNAs to Cas9 nuclease.

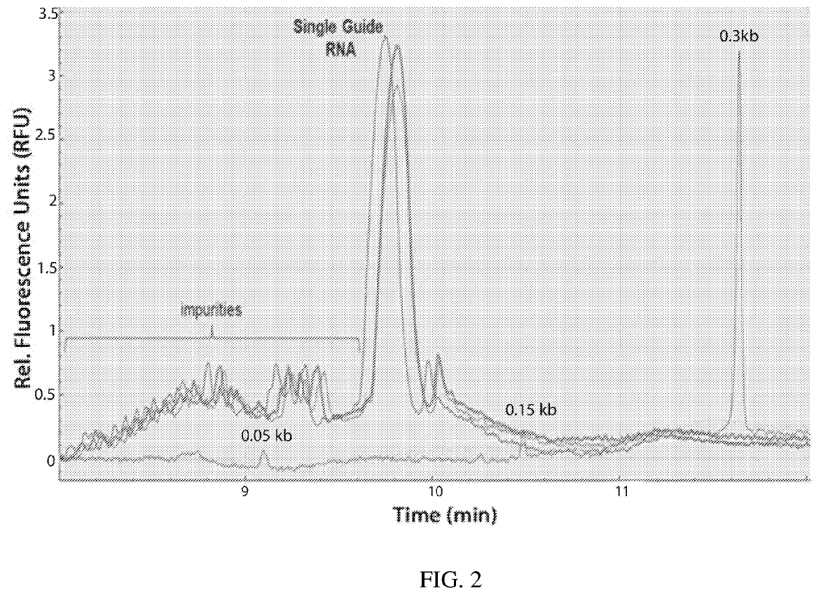

FIG. 2 shows the overlay of three injections (red, green, and blue traces) of a sgRNA sample analyzed using a method of this disclosure. The pink trace shows the ssRNA ladder and corresponding sizes. The background signal is indicated by the aqua trace by using water. The sgRNA peak(s) (100 nt) appeared between the 50 and 150 ssRNA molecular markers.

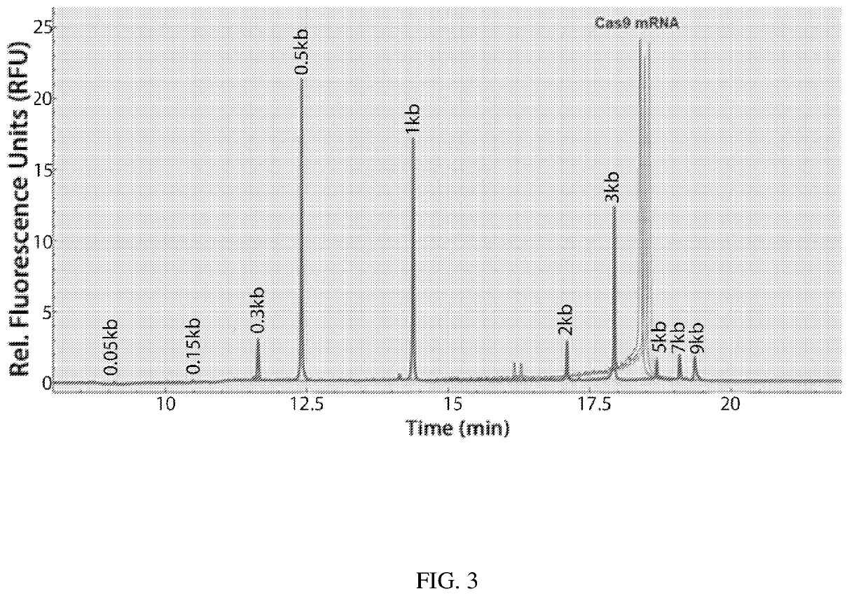

FIG. 3 shows the overlay of three injections of Cas 9 mRNA sample (red, green, and blue traces) analyzed using a method of this disclosure. The pink trace shows the ssRNA ladder and corresponding sizes. The Cas9 mRNA peak(s) (˜4.5 kb) appeared between the 3,000 and 5,000 ssRNA molecular markers.

Table 1 shows the intra-capillary corrected area % (CPA %) measurement equivalent to the nucleic acid products shown in FIG. 3. Measurements were based on five consecutive injections for a sample preparation as described. The nucleic acid impurities accounted for an average corrected area % of 26.56, the main sgRNA product of 57.97, and high molecular weight (HMW) content of 15.48. The relative low standard deviation calculations for these measurements demonstrate the repeatability performance of the disclosed methods.

TABLE 1
BioPhase 8800 intra-capillary corrected peak area
% (CPA %) of major species in the single guide RNA

| | | Main product | |
|---|---|---|---|
| sgRNA (100, nt) | Impurity CPA % | CPA % | HMW CPA % |
| Injection 1 | 28.08% | 57.97% | 13.96% |
| Injection 2 | 25.55% | 57.62% | 16.84% |
| Injection 3 | 27.68% | 57.96% | 14.36% |
| Injection 4 | 25.83% | 58.47% | 15.70% |
| Injection 5 | 25.65% | 57.83% | 16.52% |
| Average | 26.56% | 57.97% | 15.48% |
| Standard Deviation | 1.22 | 0.31 | 1.28 |
| % CV | 4.59% | 0.54% | 8.27% |

CPA %: corrected area percentage

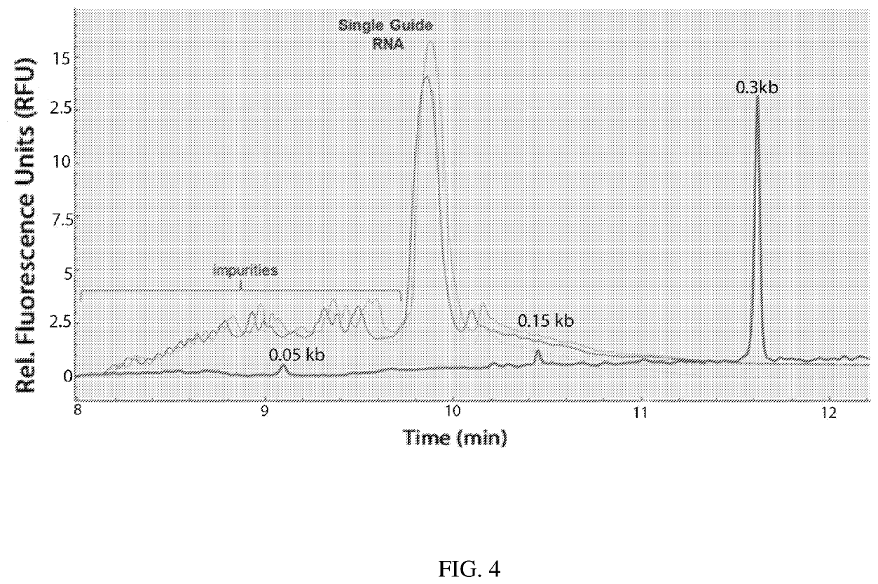

FIG. 4 shows the overlay of three injections of the sgRNA sample (red, green, and aqua traces). The blue trace shows the ssRNA ladder and corresponding sizes. The sgRNA peak(s) (100 nt) appeared between the 50 and 150 ssRNA molecular markers.

Table 2 shows the intra-capillary corrected area % (CPA %) measurement equivalent to the nucleic acid products shown in FIG. 4. The nucleic acid impurities accounted for an average corrected area % (CPA %) of 29.00 and a main RNA product of 71.03. The results support the disclosed methods for the qualification of large RNA fragments.

TABLE 2
BioPhase 8800 intra-capillary corrected peak area
% (CPA %) of major species in the Cas9mRNA

| Cas9mRNA | Impurity CPA % | Main product CPA % |
|---|---|---|
| Injection 1 | 29.70% | 70.30% |
| Injection 2 | 30.31% | 69.87% |
| Injection 3 | 28.27% | 71.73% |
| Injection 4 | 27.31% | 72.69% |
| Injection 5 | 29.43% | 70.57% |
| Average | 29.00% | 71.03% |
| Standard Deviation | 1.20 | 1.15 |
| % CV | 4.14% | 1.63% |

CPA %: corrected area percentage

Table 3 shows the intra-capillary corrected area % (CPA %) measurement equivalent to the nucleic acid products shown in FIG. 4. Measurements were based on three consecutive injections for a sample preparation as described. The nucleic acid impurities accounted for an average corrected area % of 26.10, the main sgRNA product of 63.04, and HMW content of 10.86. The relative low standard deviation calculations for these measurements demonstrate the repeatability performance of the disclosed methods.

TABLE 3
PA 800 Plus Pharmaceutical Analysis intra-capillary corrected
peak area % (CPA %) of major species in the single guide RNA

| | | Main product | |
|---|---|---|---|
| sgRNA (100, nt) | Impurity CPA % | CPA % | HMW CPA % |
| Injection 1 | 24.42% | 63.23% | 12.35% |
| Injection 2 | 26.69% | 62.60% | 10.70% |
| Injection 3 | 27.19% | 63.30% | 9.52% |
| Average | 26.10% | 63.04% | 10.86% |
| Standard Deviation | 1.48 | 0.39 | 1.42 |
| % CV | 5.66% | 0.61% | 13.09% |

CPA %: corrected area percentage

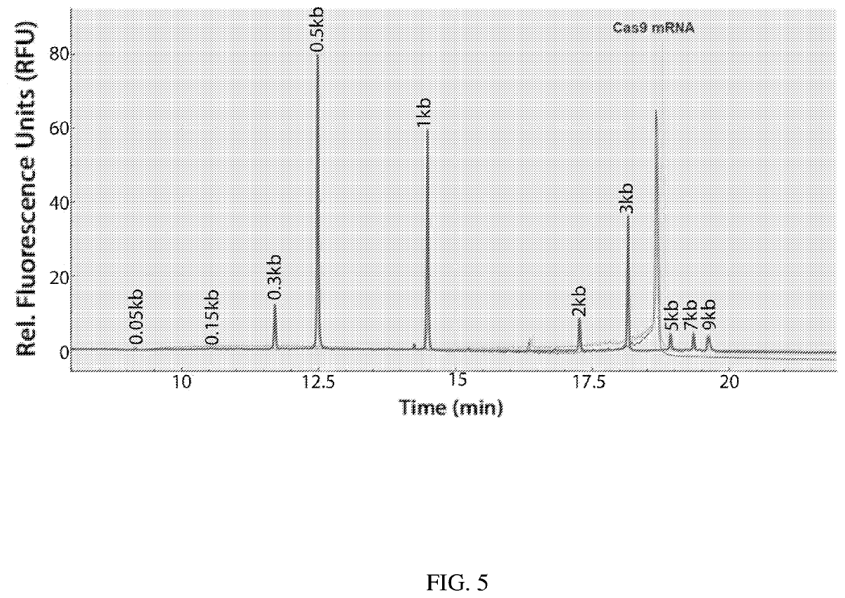

FIG. 5 shows the overlay of three Cas9 mRNA injections (red, green, and aqua traces). The blue trace shows the ssRNA ladder and corresponding sizes. The Cas9 mRNA peak(s) (˜4.5 kb) appeared closer to the 5,000 molecular marker. Consistent with the profiling using the BioPhase 8800 system and no RNA products larger than the main expected product were detected. Low level of impurities was confirmed using the PA 800 Plus Pharmaceutical Analysis system.

Table 4 shows the intra-capillary corrected area % (CPA %) measurement equivalent to the nucleic acid products shown in FIG. 5. The nucleic acid impurities accounted for an average corrected area % (CPA %) of 30.34% and a main RNA product of 69.66. The results support the use of the disclosed methods for the qualification of large RNA fragments

TABLE 4
PA 800 Plus Pharmaceutical Analysis intra-capillary corrected
peak area % (CPA %) of major species in the Cas9mRNA

| Cas9mRNA | Impurity CPA % | Main product CPA % |
|---|---|---|
| Injection 1 | 28.53% | 71.47% |
| Injection 2 | 30.96% | 69.04% |
| Injection 3 | 31.53% | 68.47% |
| Average | 30.34% | 69.66% |
| Standard Deviation | 1.59 | 1.59 |
| % CV | 5.25% | 2.29% |

CPA %: corrected area percentage

In some aspects, the method or kit may further comprise a sRNA ladder containing molecular markers for RNA fragments of about 50, about 150, about 300, about 500, about 1,000, about 2,000, about 3,000, about 5,000, about 7,000, and about 9,000 nucleotides long.

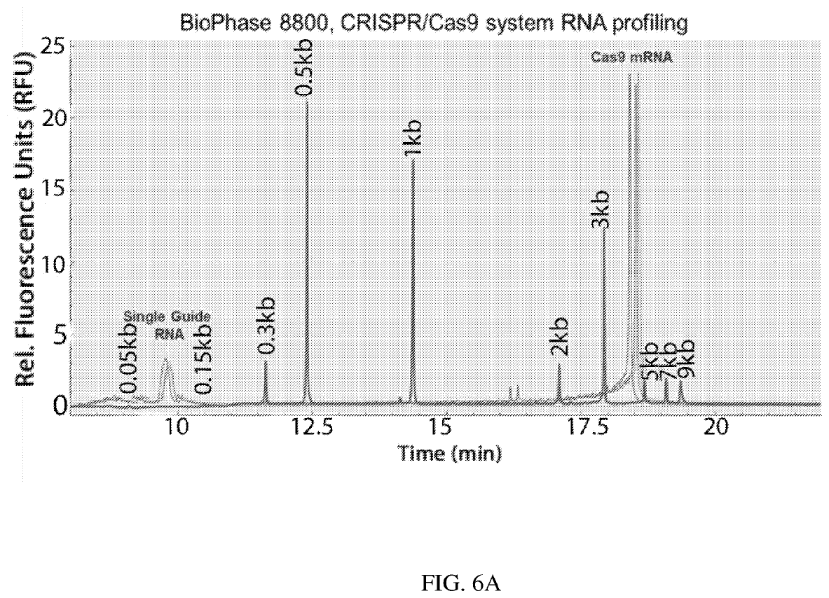

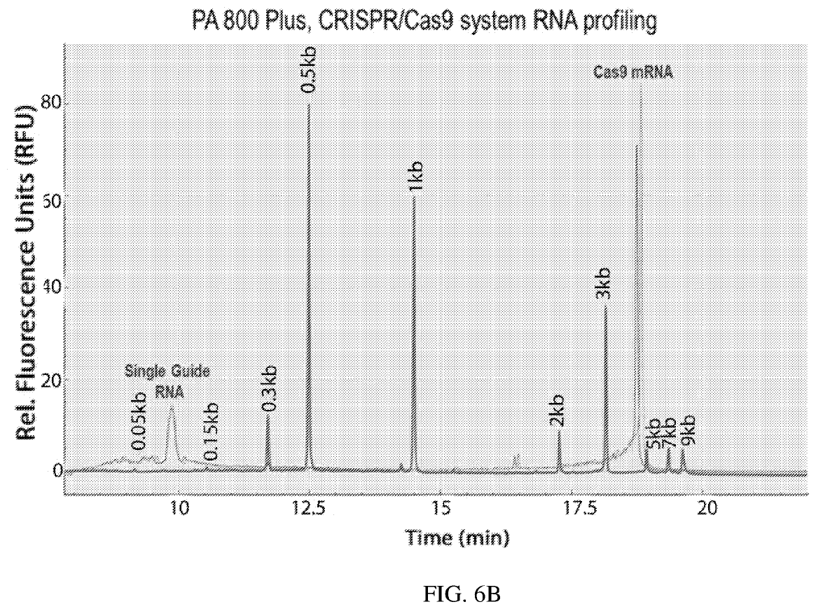

FIG. 6A illustrates an intra-capillary analysis (n=3 injections) from a mixed sample containing Cas9 mRNA and sgRNA (red, green, and blue traces). The ssRNA ladder is shown by the pink trace with the corresponding sizing for the various molecular markers. The aqua trace represents the background signal by using water. FIG. 6B illustrates an intra-capillary analysis (n=3 injections) from a mixed sample containing Cas9 mRNA and sgRNA (red, green, and aqua traces). The ssRNA ladder is shown by the blue trace with the corresponding sizing for the various molecular markers. This side-by-side nucleic acid profiling suggests the high performance and compatibility of the disclosed methods for the characterization of RNA fragments from various sizes in the range of about 50 nucleotides to about 9,000 nucleotides in length.

In another aspect, the disclosure provides a kit for analyzing at least two nucleic acids, wherein at least one nucleic acid is shorter than about 200 nucleotides, and at least one nucleic acid is longer than about 4000 nucleotides, the kit comprising a CE capillary, a cartridge comprising at least one capillary, or a capillary electrophoresis chip, a buffer comprising a polymer matrix, and instructions for use. The kit may also further comprise a fluorescent dye, a regenerating solution, a diluent, and/or an ssRNA ladder.

In another aspect, the disclosure provides for a kit for analyzing at least two nucleic acids, wherein at least one nucleic acid is shorter than about 200 nucleotides and at least one nucleic acid is longer than about 4000 nucleotides, the kit including a buffer comprising a polymer matrix and instructions for use. The kit may additionally include a fluorescent dye, a regenerating solution, a diluent, and/or an ssRNA ladder.

### EXAMPLES

### Example 1: Exemplary Sample Preparation and Analysis Method for Nucleic Acids of Differing Lengths

### Materials

The following materials and reagents were from SCIEX, Framingham, Massachusetts. RNA 9000 Purity & Integrity Kit PN, C48231) containing the Nucleic Acid Extended Range Gel, SYBR Green II RNA Gel Stain, Acid Wash (regenerating solution), CE grade water, and the ssRNA Ladder (50-9,000 nucleotides).

### Instrumentation

BioPhase 8800 system. Pre-assembled BioPhase BFS Capillary Cartridge (8 capillaries, 30 cm in total length, PN 5080121), the disposable BioPhase sample and reagent plates (PN 5080311). PA 800 Plus Pharmaceutical Analysis system: Pre-assembled Bare Fused Silica Cartridges (PN A55625) with the LIF-lens/aperture installed, PCR micro vials (PN 144709), universal vial caps (PN A62250), and universal vials (A62251), and sample loading solution (SLS, PN 608082).

The CleanCap Cas9 mRNA (5moU) (PN, L-7206) with an expected size of 4,521 nucleotides characterized by agarose gel mobility and shipped at 1.0 mg/mL was obtained from TriLink Biotechnologies, San Diego, California.

A 20 nucleotide-long proprietary single guide RNA sequence targeting the human HPRT1 gene (NM-000194) was fused by synthesis to the tracrRNA sequence at 10 nmol by Integrated DNA Technologies (IDT), Coralville, Iowa. This Alt-R CRISPR-Cas9 sgRNA product at IDT added chemical modifications to this long sgRNA (100 nucleotides; 32,458.9 g/mole) to increase the sgRNA stability, potency, and resistance against nuclease activity.

### Sample Preparation

The Cas9 mRNA was stored at −80° C. until the time of analysis. Working samples were prepared at 100 ng/μL (20 μL) from the main stock at 1 mg/mL by using CE grade water. The lyophilized sgRNA was resuspended at 3.246 μg/μL (100 μM) by using a solution of 10 mM Tris, pH 7.5, 0.1 mM EDTA, aliquoted into small volumes, and stored at −80° C. until the time of analysis. The sgRNA working solution was prepared by diluting the stock solution (3.25 μg/μL) down to 100 ng/μL (100 μL) by using CE grade water.

Cas9 mRNA Sample Analysis

The PA 800 Plus Pharmaceutical Analysis System was prepared as follows: install LIF optics; inspect and clean manifold block, electrodes, and/or injectors; install BFS capillary cartridge with LIP aperture; perform LIF calibration to set calibration correction factor (CCF); set to 15 RFU target for 50 μm ID capillary; run conditioning method at 20° C. capillary temperature; run sample separation methods at 200 V/m and 30° C. capillary temperature; run shut down method at 20° C. capillary temperature; and store capillary at 2-8° C.

sgRNA Sample Analysis

A 25 ng/μL sgRNA sample was prepared by diluting 12.5 μL of the 100 ng/μL sgRNA working solution after thawing on ice by using 37.5 μL of 50% CE grade water and 50% SLS mixture, for a total volume of 50 μL.

Cas9 mRNA and sgRNA Sample Analysis

For the co-analysis of the Cas9 mRNA and the sgRNA, 5 μL of the 100 ng/μL Cas9 mRNA working solution and 12.5 μL of the 100 ng/μL sgRNA working solution were added to 32.5 μL of a 50% CE grade water and 50% SLS mixture for a total volume of 50 μL.

After the above mixture was prepared, heat denaturation was accomplished by using a thermal cycler at 70° C. for five minutes, followed by snap cooling by placing the sample on ice until the time of injection for CE analysis. This sample preparation protocol was performed in triplicate (n=3) for the BioPhase 8800 analysis and injected five times for repeatability assessment. Single preparation was performed for the PA 800 Plus Pharmaceutical Analysis system; however, the sample was injected three times for repeatability analysis.

ssRNA Ladder Preparation

The ssRNA ladder was prepared according to the RNA 9000 Purity & Integrity Kit application guide. A 50 μL sample was denatured by using a thermal cycler at 70° C. for five minutes, followed by snap cooling by placing the sample on ice until the time of injection for CE analysis. The number of prepared ssRNA ladder samples depended on a number of replicates needed.

### CE Separation Method

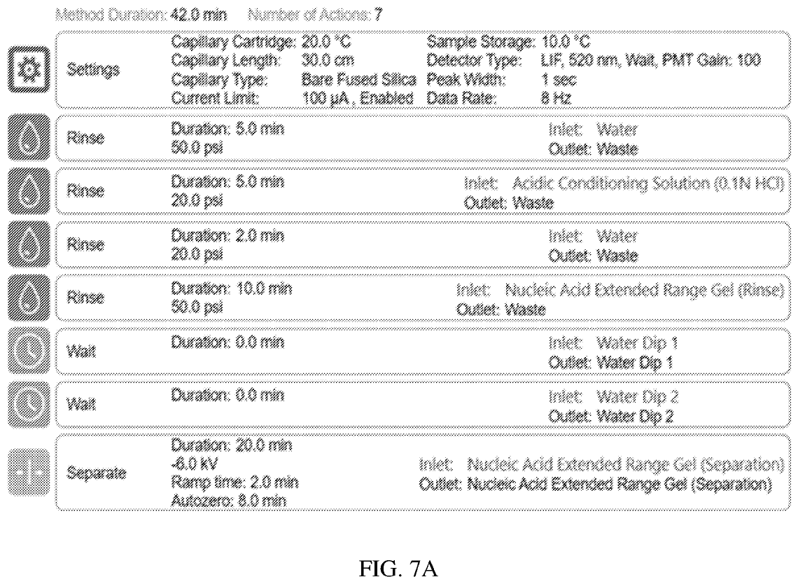

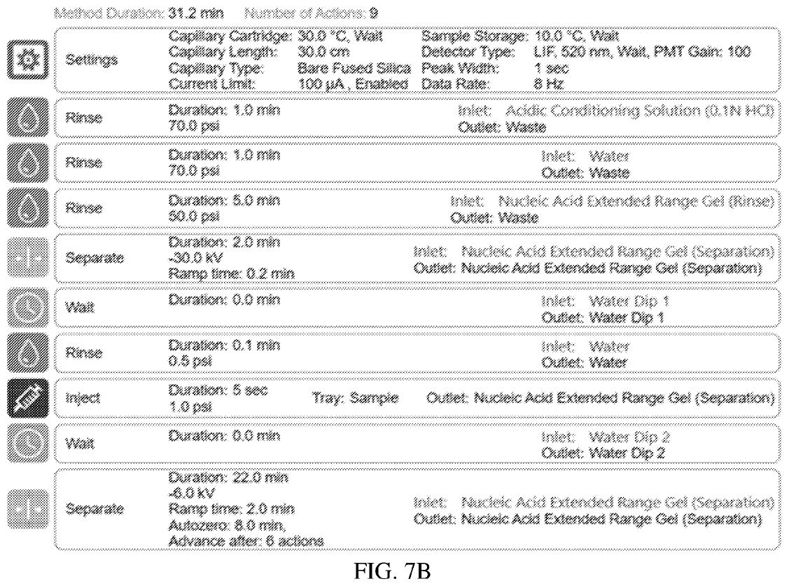

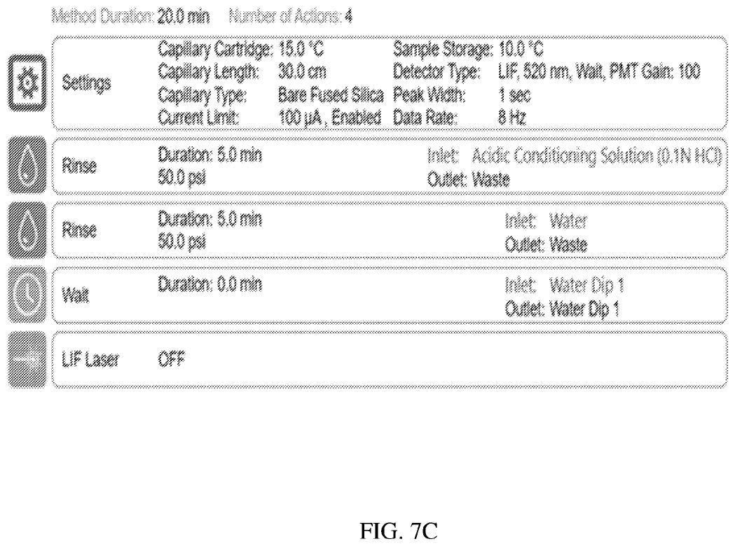

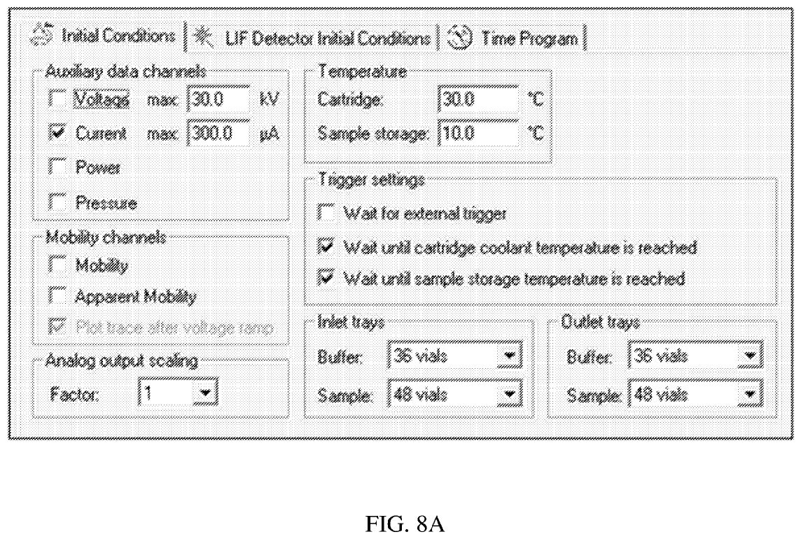

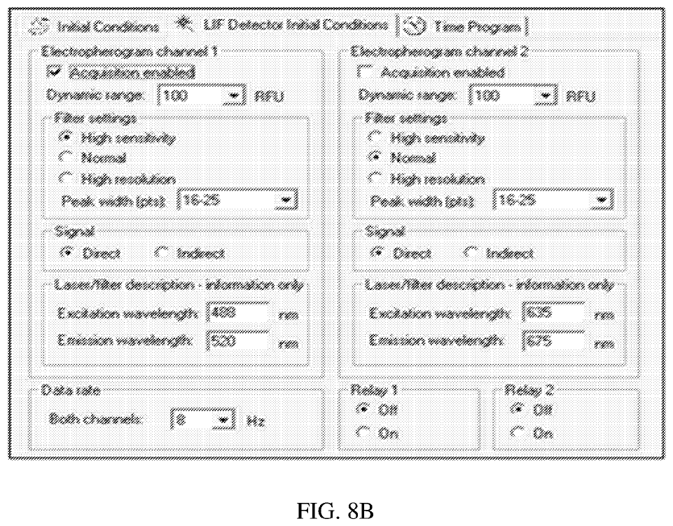

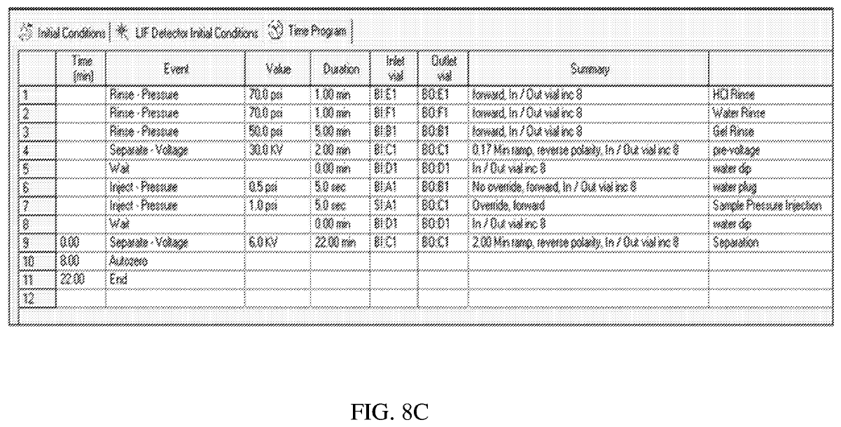

FIGS. 7A-7C illustrate the BioPhase 8800 system operation sequence for the conditioning (FIG. 7A), separation (FIG. 7B), and shut down methods (FIG. 7C) used for this study. FIGS. 8A-8C shows the transferability of this pressure-based separation method into the PA 800 Plus Pharmaceutical Analysis system.

### Data Processing

The BioPhase Software 1.0 was used to calculate corrected area % (CPA %) for the main product, nucleic acid impurities, and higher molecular weight (HMW's) species. A 0.75% positive threshold, with suspended integration from 0 to 8 minutes was applied throughout the analysis of this study. PA 800 Plus Pharmaceutical Analysis data files were exported as ASCII files (.asc) in 32Karat software for BioPhase Software 1.0 analysis. Values were tabulated on a spreadsheet software program (Microsoft Excel) to calculate the average, standard deviation, and the percent coefficient variation.

### Example 2: Exemplary Sample Preparation and Analysis Method for Samples Containing Small or Large ssRNA Fragments

### Materials

Unless otherwise noted, the materials listed below are purchased from SCIEX. RNA 9000 Purity & Integrity Kit (PN, C48231) containing the nucleic acid extended range gel comprising SYBR Green II RNA gel stain, acid wash (regenerating solution), CE grade water, the RNA ladder (50-9,000 nucleotides) and LIF calibration solution. Pre-Assembled BFS Capillary Cartridge (30.2 cm bare-fused silica capillary, PN A55625). The SCIEX universal vials (P/N A62251), universal vial caps (P/N A62250) and PCR vials (P/N 144709) were used for sample and reagent loading.

### Sample Preparation

The RNA 9000 molecular ladder (50-9,000 nucleotides) was diluted with SLS as described in the user's manual. Specifically, mix 4 μL of ssRNA ladder in the kit with 96 μL of sample loading solution, heat the mixture for five minutes at 70° C. using a thermal cycler, and immediately cooled on ice for at least ten minutes. Transfer 80 μL of the sample into the sample vial, before subject to sample analysis.

### Instrument and Software

PA 800 Plus pharmaceutical analysis CE system (SCIEX) equipped with a laser-induced fluorescence (LIF) detector with a 488 nm solid state laser and a 520 nm emission filter was used for all separations. The LIF detector was calibrated according to the user guide. Data acquisition was performed using 32 Karat software version 10 and exported to ASCII format, later imported and processed using the BioPhase Software for integration and calculations including Signal intensity, Corrected Area, and Corrected Area %.

### Instrument Set Up

The preparation of the separation gel consists of mixing 10 μL of SYBR Green II RNA gel stain in the kit with 5 mL of Nucleic acid extended range gel for eight injections. This separation gel preparation can be scaled according to the number of samples to be analyzed. However, it must be prepared at the time of the experiment. Any leftover separation gel must be discarded accordingly. Add 1.5 mL of separation gel and all other reagents needed for this application into the PA 800 plus universal vials and load onto the instrument according to the reagent plate map provided in the user guide. The separation method can be downloaded from Sciex.com or created following the user guide. For separation temperature screening, the capillary cartridge temperature can be set ranging from 25 to 55° C. for the pressure injection methods.

### Effect of Separation Temperature

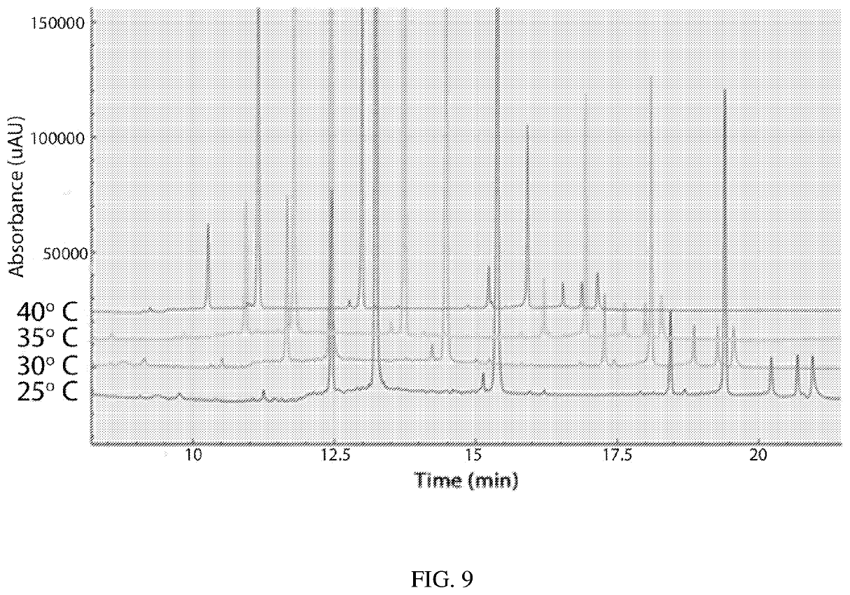

Separation temperature is one of the critical components for capillary electrophoresis method development. Higher temperatures can speed up the run time and affect peak resolution and peak shape. The PA 800 plus system provides an accurate temperature control system allowing the user to set, evaluate and control the separation temperature to achieve an optimal balance of assay throughput and separation efficiency. The separation of the ssRNA ladder with a size range from 0.05 to 9 kilonucleotides (“knt” or “kb”) as a function of the temperature was assessed. The overlaid electropherogram shown in FIG. 9, illustrates a decrease in separation time as the separation temperature increases. An expected behavior since the temperature reduces the viscosity of the sieving matrix of the separation gel. Notably, the separation temperature also affected the resolution between the ssRNA markers. However, the change in resolution is dependent on the ssRNA fragment size. Higher temperature improves resolution on the larger RNA while lower temperature improves the resolution on the lower-sized marker as shown in Table 5. Table 5 is a summary table of assay performance criteria including capillary run life, total assay time (9 kb marker MT) resolution between 7 and 9 kb and theoretical plates. For each temperature, multiple capillaries were evaluated.

TABLE 5

| | | Migration | Resolution | | | | |
|---|---|---|---|---|---|---|---|
| Capillary | Expected | Time of 9 | between 7 | EP Theoretical Plates (×10⁶) | | | |
| Temperature | Capillary | kb Peak | and 9 kb | Peak | | | |
| (° C.) | Run Life | (min) | peaks | 1 kb | 5 kb | 7 kb | 9 kb |
| 25 | 130-150 | 21.18 | 3.62 | 1.19 | 1.37 | 1.67 | 1.11 |
| 30 | 100-120 | 19.78 | 4.31 | 1.22 | 1.98 | 1.87 | 1.12 |
| 35 | 70-80 | 18.81 | 5 | 1.24 | 2.02 | 1.92 | 1.39 |
| 40 | 50-60 | 17.35 | 5.1 | 1.22 | 1.95 | 1.78 | 1.39 |

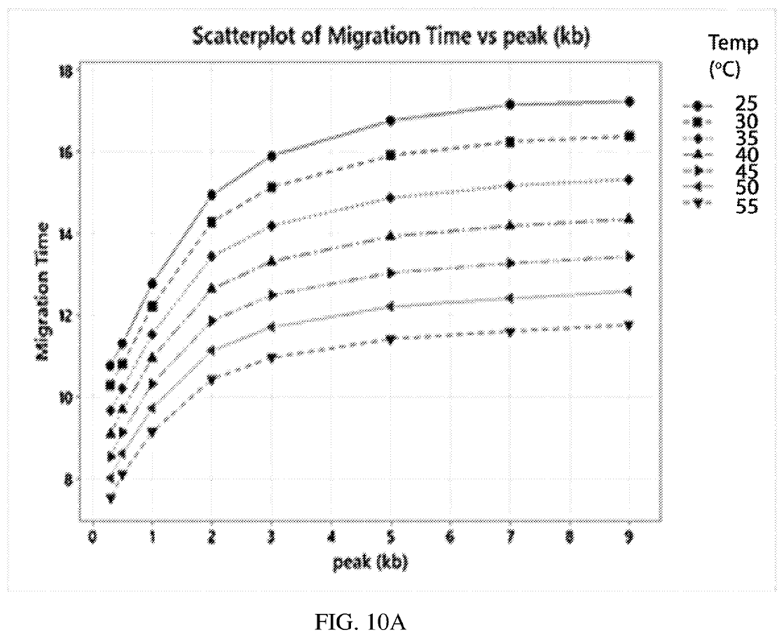

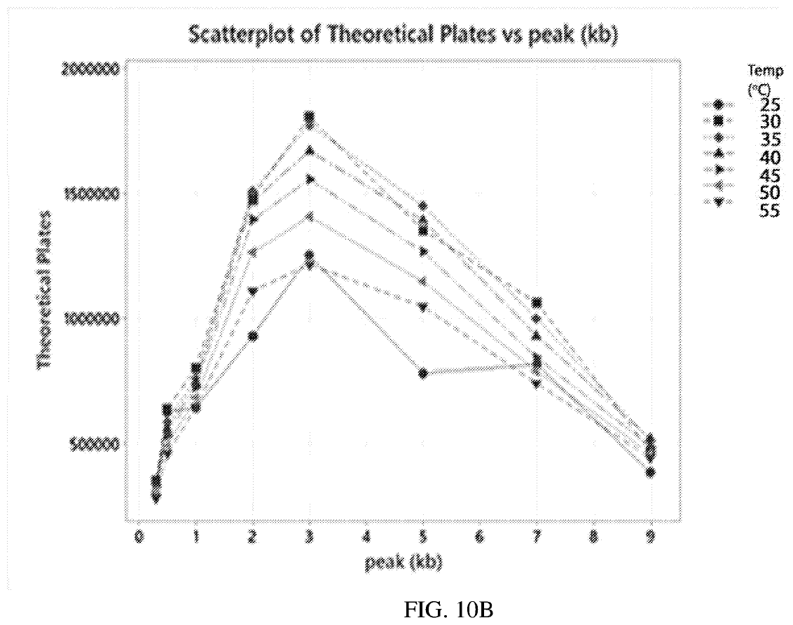

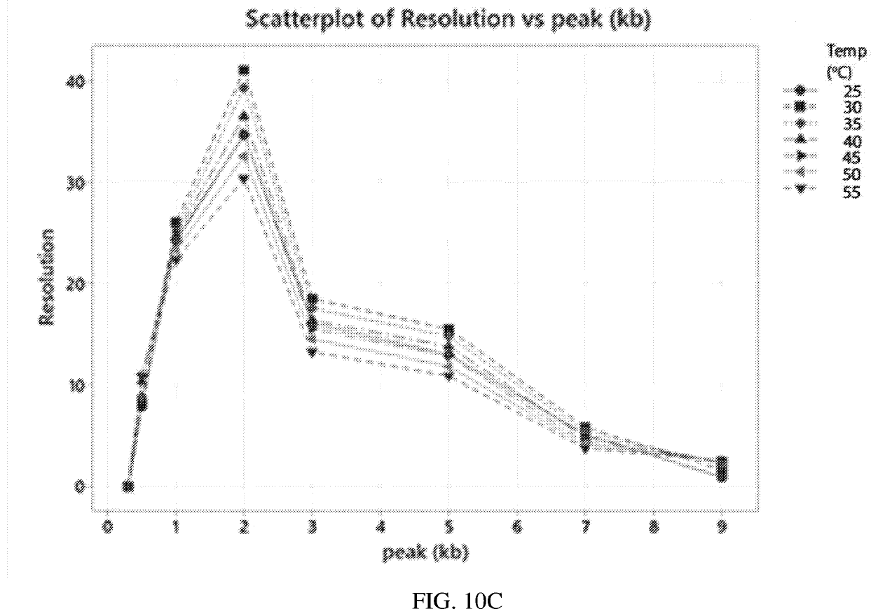

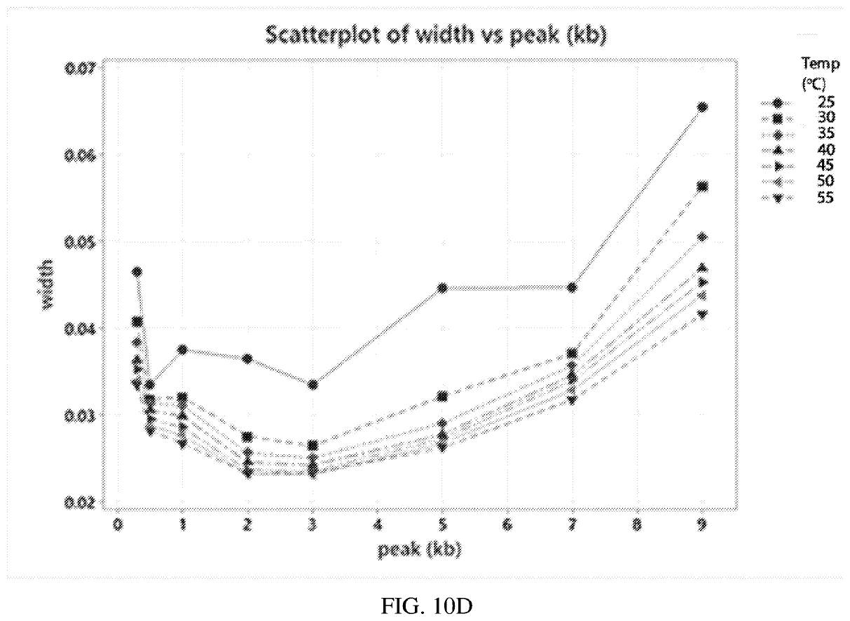

FIGS. 10A-10D depict the scatter plot of migration time, theoretical plates, and peak width for each of the ssRNA markers as a function of the separation temperature.

Interestingly, it was found that increased separation temperature may detrimentally impact separation performance over time. Multiple cartridges were evaluated in the temperature range of 25-40° C. to establish the safest operation limit for capillary longevity. Table 5 summarizes the results that revealed 30° C. is the best separation temperature for both the run life of the capillary and resolution of fragment sizes of therapeutic interest, which lies between 1 kb and 9 kb.

### Evaluation of Injection Modes

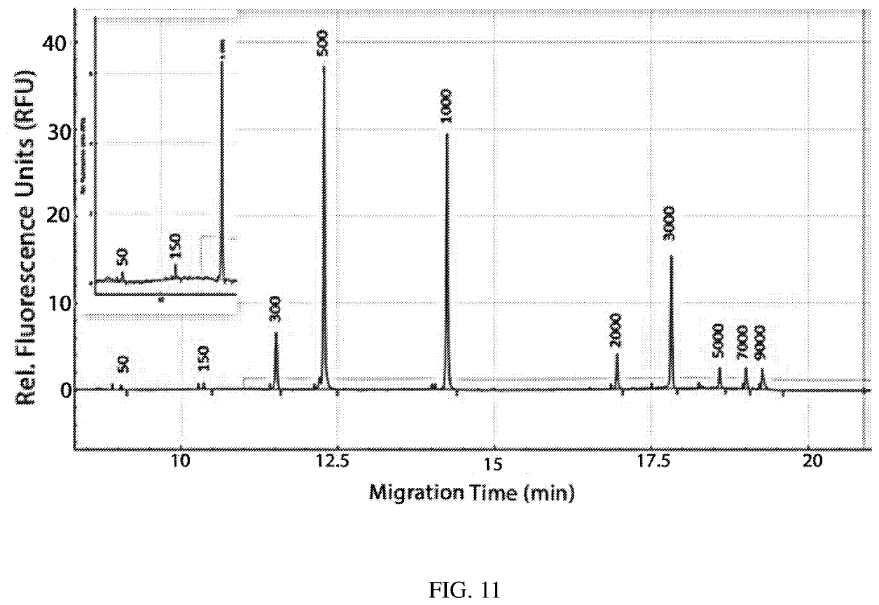

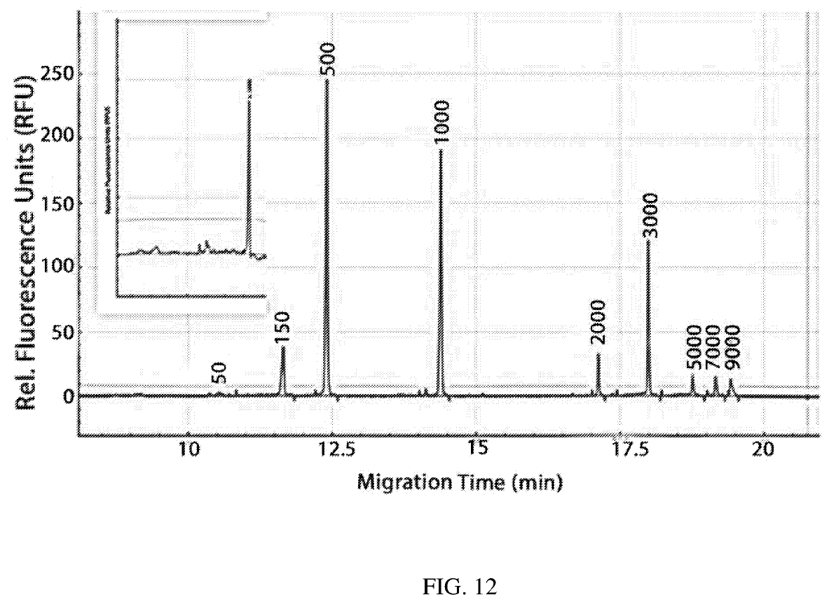

Two types of injection can be used with the RNA 9000 Purity & Integrity Kit, hydrodynamic injection or HDI and electrokinetic injection or EKI. Briefly, HDI is where pressure is used to drive a small volume of sample into the capillary. EKI is where an electric field is used to drive only the charged species of the sample into the capillary. This case uses reverse polarity in CGE where residual electro-osmotic (EOF) flow moves away from the detector, introducing only anionic species into the capillary. The difference between these two injections is that the HDI is representative of all sample components, whereas the EKI injection introduces a bias because it preferentially injects species in the sample with higher mobility. FIGS. 11 and 12 depict the typical profile of ssRNA ladder using HY and EK injections, respectively.

The results indicated that both HDI and EKI have similar profiles for markers above 300 nucleotides. Interestingly, an observable increased peak widths of fragments between 50 and 300 nucleotides is seen when using the EKI, and which is not observed in pressure injection. This results in decreased theoretical plates and could have a negative impact on the purity assessment of fragments having a size between 50 and 150 nucleotides. Therefore, a pressure injection should be considered if the analyte of interest is in the size range of below 300 nucleotides. Conversely, EKI is inherently a stacking technique because it induces a pre-concentration of the sample band during the injection, resulting in a five-fold increase in signal response. Furthermore, EKI injection is recommended when working with samples with low concentrations and containing RNA fragments above 300 nucleotides.

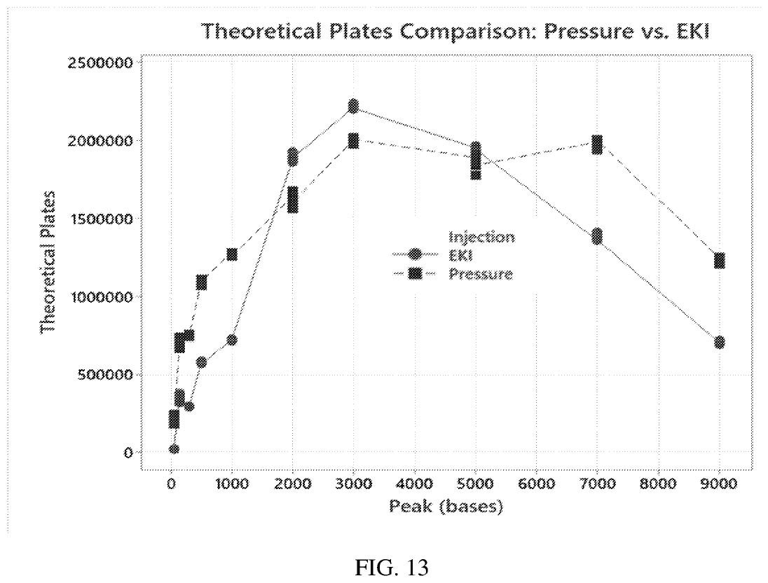

It is possible to impart stacking capability to the HDI by injecting a small water plug before the injection of the sample. This technique allows for a significant improvement of peak shape and resolution for the ssRNA fragments smaller than 300 nucleotides. A quantitative evaluation on the theoretical plates between EKI and HDI is shown in FIG. 13. Therefore, HDI is recommended for analytes with a size below 500 nucleotides.

### Assay Repeatability

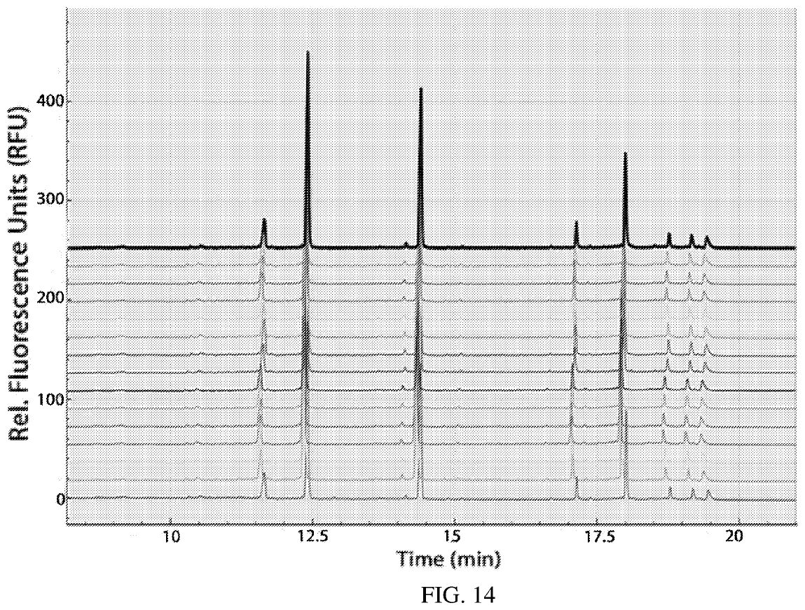

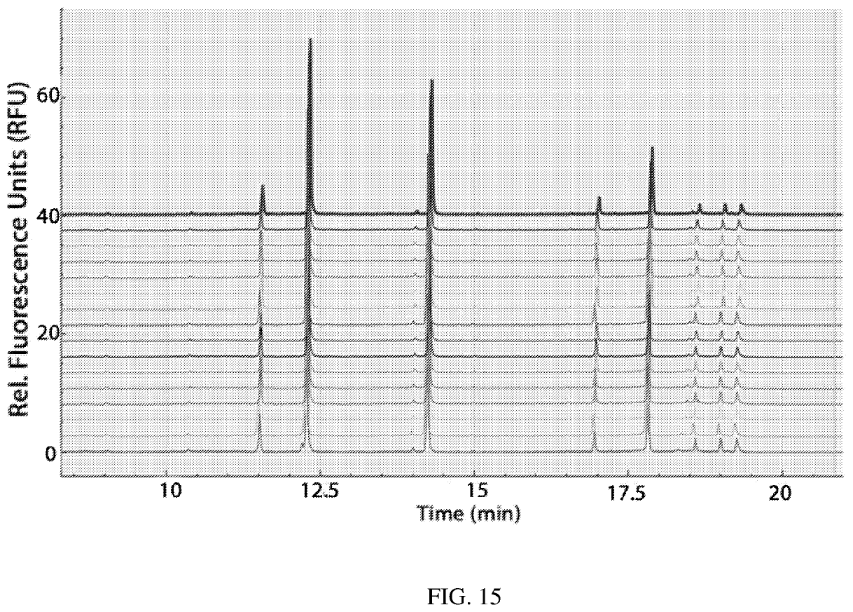

The repeatability of the method for each injection mode at 30° C. separation temperature was evaluated. Sixteen injections were performed for each freshly prepared sample, using EKI (FIG. 14) and HDI (FIG. 15) and the average and % RSD for migration time and corrected peak area was calculated for each fragment size marker peak. Only fragments above 300 nucleotides were considered in the calculations for the EKI (Table 6), while all ten fragments markers were evaluated for the hydrodynamic injections (Table 7). Table 6 shows the repeatability evaluation of electrokinetic injection (EKI) for the separation of ssRNA ladders. Table 7 repeatability evaluation of hydrodynamic injection (HDI) for the separation of ssRNA ladders.

TABLE 6

| Peak ID | [300] | [500] | [1000] | [2000] | [3000] | [5000] | [7000] | [9000] |
|---|---|---|---|---|---|---|---|---|
| Average for MT | 11.62 | 12.38 | 14.37 | 17.10 | 17.96 | 18.72 | 19.12 | 19.39 |
| % RSD for MT | 0.23 | 0.20 | 0.18 | 0.18 | 0.19 | 0.19 | 0.19 | 0.19 |
| Average for % | 8.96 | 40.46 | 29.15 | 3.04 | 11.93 | 2.62 | 1.91 | 1.93 |
| Corrected Area | | | | | | | | |
| % RSD for | 7.20 | 1.54 | 2.30 | 7.49 | 4.00 | 9.62 | 10.99 | 6.42 |
| Corrected Area | | | | | | | | |

TABLE 7

| Peak ID | [50] | [150] | [300] | [500] | [1000] | [2000] | [3000] | [5000] | [7000] | [9000] |
|---|---|---|---|---|---|---|---|---|---|---|
| Average for MT | 9.03 | 10.38 | 11.54 | 12.31 | 14.27 | 16.98 | 17.84 | 18.61 | 19.02 | 19.28 |
| % RSD for MT | 0.09 | 0.15 | 0.15 | 0.15 | 0.15 | 0.13 | 0.13 | 0.13 | 0.13 | 0.13 |
| Average for % | 0.48 | 0.54 | 7.77 | 40.59 | 30.07 | 3.10 | 11.70 | 2.15 | 1.51 | 2.09 |
| Corrected Area | | | | | | | | | | |
| % RSD for | 10.56 | 4.82 | 1.86 | 1.43 | 1.74 | 1.44 | 1.24 | 4.24 | 2.00 | 2.71 |
| Corrected Area | | | | | | | | | | |

When EKI injection is used, the fragment markers migration time overall increased slightly (FIG. 14), % RSD for migration time is below 0.25%). Also, due to the small contribution of 50 and 150 nucleotides for the total corrected area, the reported % CA for markers above 300 nucleotides are not affected when different injection modes are used. The separations using EKI injection show a higher % RSD for corrected peak area percentage for each marker, possibly due to the inherent bias introduced by electrokinetic injection. It is worth noting that the average total corrected peak area for pressure injection is 4.52, compared to 34.99 for the EKI injection, consistent with the increased sample load typical of an EKI injection.

While the present disclosure has been described with reference to certain embodiments, it will be understood by those skilled in the art that various changes may be made and equivalents may be substituted without departing from the scope of the present disclosure or appended claims. In addition, many modifications may be made to adapt a particular situation or material to the teachings of the present disclosure without departing from its scope. Therefore, it is intended that the present disclosure not be limited to the particular embodiments disclosed, but that the present disclosure will include all aspects falling within the scope of the appended claims.

All patents, patent applications, publications, and descriptions mentioned above are herein incorporated by reference in their entirety.

What is claimed is:

1. A method for analyzing nucleic acids of differing sizes, the method comprising:
loading at least two nucleic acids of differing sizes on a capillary electrophoresis (CE) capillary wherein the CE capillary is filled with a buffer comprising a polymer matrix;
applying a voltage to the CE capillary to simultaneously separate the nucleic acids; and
detecting the separated nucleic acids with a detector, wherein at least one nucleic acid is shorter than about 200 nucleotides and at least one nucleic acid is longer than about 4000 nucleotides.

2. The method of claim 1, wherein the at least two nucleic acids are loaded onto the CE capillary using hydrodynamic injection or electrokinetic injection.

3. The method of claim 2, wherein the at least two nucleic acids are loaded onto the CE capillary using hydrodynamic injection

4. The method of any one of the preceding claims, wherein at least one nucleic acid is shorter than about 150 nucleotides, alternatively shorter than about 100 nucleotides, alternatively shorter than about 50 nucleotides, alternatively shorter than about 25 nucleotides.

5. The method of any one of the preceding claims, wherein at least one nucleic acid is longer than about 4200 nucleotides, alternatively longer than about 4500 nucleotides, alternatively longer than about 4800 nucleotides, alternatively longer than about 5000 nucleotides, alternatively longer than about 5500 nucleotides, alternatively longer than about 6000 nucleotides, alternatively longer than about 6500 nucleotides, alternatively longer than about 7000 nucleotides, alternatively longer than about 7500 nucleotides, alternatively longer than about 8000 nucleotides, alternatively longer than about 8500 nucleotides, alternatively longer than about 9000 nucleotides, alternatively longer than about 9500 nucleotides, alternatively longer than about 10000 nucleotides.

6. The method of any one of the preceding claims, wherein the at least two nucleic acids are selected from the group consisting of deoxyribonucleic acid (DNA), ribonucleic acid (RNA), single-stranded DNA (ssDNA), double-stranded DNA (dsDNA), microRNA (miRNA), messenger RNA (mRNA), and RNA fragments.

7. The method of any one of the preceding claims, wherein the at least two nucleic acids are single stranded RNA fragments.

8. The method of claim 7, wherein the single stranded RNA fragments are sgRNA and Cas9mRNA.

9. The method of any one of the preceding claims, wherein the at least two nucleic acids are loaded on the CE capillary as a mixture or are loaded sequentially.

10. The method of any one of the preceding claims, wherein the polymer matrix is selected from the group consisting of crosslinked polymer, linear polymers, slightly branched polymers, linear polyacrylamide, polyethylene oxide, polyethylene glycol and dextran.

11. The method of any one of the preceding claims, further comprising adding a fluorescent dye to the at least two nucleic acids, the polymer matrix and/or to a buffer disposed within the CE capillary, wherein the fluorescent dye binds the nucleic acids resulting in fluorescently labeled nucleic acids.

12. The method of claim 11, wherein the fluorescent dye is a cyanine-based dye.

13. The method of claim 12, wherein the cyanide-based dye is selected from the group consisting of Cy2, Cy3, Cy3B, Cy3.5, Cy5, Cy5.5, Cy7, SYBR Green I, SYBR Green II, PicoGreen, Thiazole orange, and Oxazole yellow.

14. The method of any one of the preceding claims, further comprising heating at least one of the at least two nucleic acids prior to loading the nucleic acids on the CE capillary.

15. The method of claim 14, wherein the nucleic acid is heated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C.

16. The method of claim 14 or claim 15, wherein the nucleic acid is heated for at least 2 minutes, alternatively at least 3 minutes, alternatively at least 4 minutes, alternatively at least 5 minutes.

17. The method of any one of claims 14-16, further comprising cooling the nucleic acid after heating.

18. The method of claim 17, wherein the nucleic acid is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

19. The method of any one of the preceding claims, wherein at least one of the at least two nucleic acids is diluted with a sample solution, water, or combinations thereof prior to loading on the CE capillary.

20. The method of any one of the preceding claims, wherein the at least two nucleic acids are separated using capillary gel electrophoresis or capillary electrochromatography.

21. The method of any one of the preceding claims, wherein the detector is a UV detector or fluorescence detector.

22. The method of claim 21, wherein the detector is a laser-induced fluorescence (LIF) detector, a lamp-based fluorescence detector, or a native fluorescence detector.

23. The method of any one of the preceding claims, wherein detecting the nucleic acid utilizes a fluorescence detector.

24. The method of any one of the preceding claims, wherein the method results in increased peak efficiency and/or high-resolution.

25. A kit for analyzing at least two nucleic acids, wherein at least one nucleic acid is shorter than about 200 nucleotides and at least one nucleic acid is longer than about 4000 nucleotides, the kit comprising:
a CE capillary, a cartridge comprising at least one capillary, or a capillary electrophoresis chip,
a buffer comprising a polymer matrix,
and instructions for use.

26. The kit of claim 25, wherein the kit further comprises a fluorescent dye, a regenerating solution, a diluent, and/or an ssRNA ladder.

27. A kit for analyzing at least two nucleic acids, wherein at least one nucleic acid is shorter than about 200 nucleotides and at least one nucleic acid is longer than about 4000 nucleotides, the kit comprising a buffer comprising a polymer matrix and instructions for use.

28. The kit of claim 27, wherein the kit further comprises a fluorescent dye, a regenerating solution, a diluent, and/or an ssRNA ladder.
